(19) United States
(12) Plant Patent Application Publication
(10) Pub. No.: US 2017/0367230 P1
(43) Pub. Date: Dec. 21, 2017

(54) Apple Tree Named 'Luiza'

(50) ;

(71) Applicant: Empresa de Pesquisa Agropecuaria e Extensáo Rural de Santa Catarina (Epagri), Florianopolis (BR)

(72) Inventors: Frederico Denardi, Cacador (BR); Marcus Vinicius Kvitschal, Cacador (BR); Maraisa Crestani Hawerroth, Cacador (BR)

(73) Assignee: Empresa de Pesquisa Agropecuaria e Extensáo Rural de Santa Catarina (Epagri), Florianopolis (BR)

(21) Appl. No.: 14/999,705

(22) Filed: Jun. 15, 2016

(51) Int. Cl. A01H 5/08

(52) U.S. Cl. CPC A01H 5/0875
USPC PLT/161

(57) ABSTRACT

‘Luiza’ apple tree (Malus domestica Borkh) is a new variety selected for its low chilling requirement, resistance to Gala leaf spot disease, high fruit quality, and early fruit maturity.

Latin name: Malus domestica.

Variety denomination: ‘Luiza’.

### BACKGROUND OF THE VARIETY

‘Luiza’ is a new and distinct variety of apple tree (Malus domestica Borkh) obtained from a controlled cross of ‘Imperatriz’ (female parent, not patented)×‘Cripps Pink’ (male parent, U.S. Plant Pat. No. 7,880) carried out at Caçador, Santa Catarina, Brazil in 2001. Seeds obtained from the cross were planted at Caçador, and ‘Luiza’ was selected from the resulting seedlings for propagation and further observation. ‘Luiza’ was first asexually propagated by grafting in 2004 at Caçador, and has since been observed to remain true to type over successive asexually propagated generations.

### BRIEF DESCRIPTION OF THE VARIETY

‘Luiza’ apple tree is distinguished by its resistance to Gala leaf spot disease (Colletotrichum spp.) and its low chilling requirement. Fruit of ‘Luiza’ is notable for its high quality, having crisp juicy flesh and a high sugar content. Fruit of ‘Luiza’ ripens earlier than comparable varieties.

‘Luiza’ is distinguished from female parent ‘Imperatriz’ by its earlier ripening; more intense skin color; and higher sugar content.

‘Luiza’ is distinguished from male parent ‘Cripps Pink’ by its lower chilling requirement; earlier fruit maturity; higher level of juiciness, crispness and sugar; lower acidity; deeper red skin color; and resistance to Gala leaf spot disease.

‘Luiza’ is distinguished from standard ‘Gala’, a similar variety, by its lower chilling requirement; resistance to Gala leaf spot disease; less prominent striping and more complete and intense overcolor; and production of fruit predominantly on spur buds, as compared to ‘Gala’, which produces fruit on both spur buds and terminal buds of long shoots.

### BRIEF DESCRIPTION OF THE PHOTOGRAPHS

The accompanying photographs of the ‘Luiza’ variety were taken from 2013 to 2015 at Caçador, Brazil.

FIG. 1 is a photograph of a ‘Luiza’ apple;

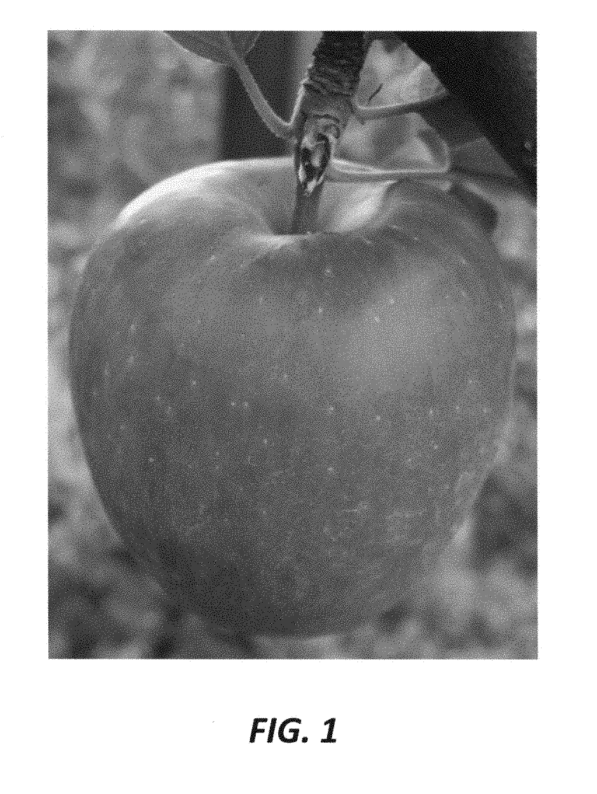

FIG. 2 is a photograph of ‘Luiza’ apple trees in bloom; and,

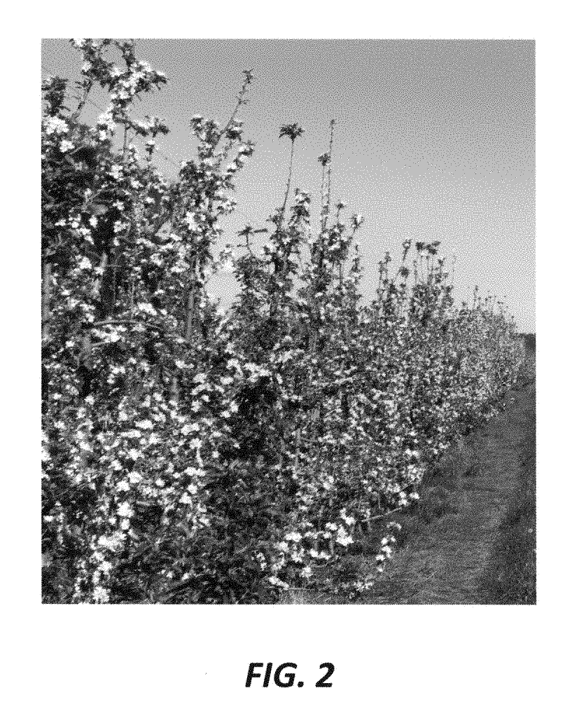

FIG. 3 is a photograph of ‘Luiza’ apple trees and fruit.

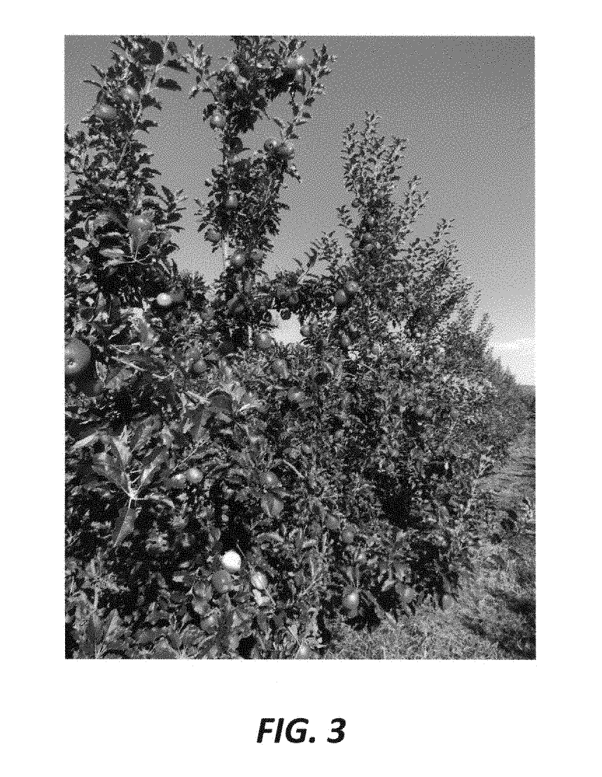

### DETAILED BOTANICAL DESCRIPTION OF THE VARIETY

The following-detailed botanical description is based on observations made at Caçador, Brazil from 2013 to 2015, of trees planted in 2011, grown on ‘Marubakaido’ rootstock (not patented) with ‘M-9’ interstock (not patented). All colors are described according to The Royal Horticultural Society Colour Chart. It should be understood that the characteristics described will vary somewhat depending upon cultural practices and climatic conditions, and will vary with location and season. Quantified measurements are expressed as an average of measurements taken from a number of individual plants of the new variety. The measurements of any individual plant or any group of plants of the new variety may vary from the stated average.
Tree:
   Vigor.—Medium.
   Type.—Ramified.
   Habit.—Spreading.
   Height.—3.8 m.
   Trunk diameter (at 30 cm above the graft).—8.8 cm.
   Bark texture.—Smooth.
   Bark color.—Yellow green 152B.
   Lenticel size.—1.4 mm.
   Lenticel color.—Yellow white 158A.
Branch (fruiting branches located at around 1 m above the graft union):
   Length.—1.3 m.
   Diameter.—2.5 cm.
   Crotch angle.—70° to 80°.
   Bark color.—Greyed-green 198A.
   Lenticel length.—1.2 mm.
   Lenticel color.—Greyed-yellow 162A.
   Number of lenticels per cm².—5.4.
One year old shoot:
   Length.—0.8 m.
   Color.—Greyed-brown 199B.
   Pubescence.—Weak.
   Thickness.—6.7 mm.
   Internode length.—2.4 cm.
   Number of lenticels per cm².—2.7.
Flower buds:
   Quantity per spur.—1.6.
   Shape.—Spiky.
   Length.—8.8 mm.
   Diameter.—4.5 mm.
   Color.—Greyed-orange 166A.
   Date of first bloom.—September 10 to 18.
   Date of full bloom.—September 25 to October 3.
   Pollination requirement.—Yes.
Leaves:
   Shape.—Ovate.
   Length.—10.3 cm.
   Width.—5.4 cm.
   Length/width.—ratio 1.9.
   Blade margin.—Serrate.
   Apex shape.—Acute.
   Base shape.—Rounded.
   Color of upper surface Green.—N137A.
   Color of lower surface.—Yellow-green 147C.
   Attitude in relation to shoot.—Upward.
Petiole:
   Length.—27 mm.
   Diameter.—2 mm.
   Color.—Yellow-green 146C.
Fruit:
   Quantity per cluster.— [symbol] .
   Diameter.—69 mm.
   Height.—69 mm.
   Weight.—170 g.
   Ratio of height to width.—1.
   General shape in profile.—Conical.
   Position of maximum diameter.—On the third near to the stalk cavity.
   Ribbing.—Medium.
   Crowning at calyx end.—Medium.
   Size of eye.—6 mm.
   Aperture of eye.—8 mm.
   Length of sepal.—7 mm.
   Bloom of skin.—Absent to weak.
   Greasiness of skin.—Absent to weak.
   Background color of skin.—Yellow 4A.
   Over color of skin.—Red 46A.
   Amount of over color.—70% to 80%.
   Intensity of over color.—Medium.
   Pattern of over color.—Solid flush with strongly defined stripes.
   Amount of russet around stalk cavity.—Very small.
   Amount of russet on cheeks.—Absent.
   Area of russet around eye basin.—Absent.
   Length of stalk.—20 mm.
   Thickness of stalk.—6 mm.
   Depth of stalk cavity.—11 mm.
   Width of stalk cavity.—19 mm.
   Depth of eye basin.—10 mm.
   Width of eye basin.—26 mm.
   Firmness of flesh.—17 to 18 pounds.
   Flesh texture.—Fine.
   Aroma.—Medium.
   Juiciness.—Very juicy.
   Brix.—13.0 to 13.5 ° Brix.
   Flesh color.—Yellow 11C.
   Stem color.—Yellow-green 152C.
Seeds:
   Quantity per fruit.—8.
   Shape.—Ovate.
   Color.—Brown 200B.
Harvest:
   Harvest date (actual and relative).—Early (beginning of February, in Brazil).
   Number of picks.—Two.
   Amount of fruit produced per tree per harvest.—18 to 20 Kg per plant.
Disease resistance/susceptibility: Susceptible to apple scab (Venturia inaequalis); Resistant to Gala leaf spot (Colletotrichum spp.); Tolerant to powdery mildew (Podosphaera leucotricha).
Market use: Fresh.

1. I claim a new and distinct apple tree substantially as described and illustrated herein.
